(19) United States
(12) Patent Application Publication
(10) Pub. No.: US 2016/0100965 A1
(43) Pub. Date: Apr. 14, 2016

(54) PUSHER GUIDE WIRE

(71) Applicant: ASAHI INTECC CO., LTD., Nagoya-shi (JP)

(72) Inventor: Makoto NISHIGISHI, Owariasahi-shi (JP)

(21) Appl. No.: 14/811,061

(22) Filed: Jul. 28, 2015

(30) Foreign Application Priority Data
Oct. 8, 2014 (JP) 2014-207392

(51) Int. Cl. A61F 2/95

(52) U.S. Cl. CPC A61F 2/95; A61F 2002/9505

(57) ABSTRACT

A pusher guide wire for delivering a stent to a target site includes a core shaft, a coil body that covers a distal end portion of the core shaft, a pusher portion that is fixed to the core shaft at a position proximal to the coil body, and a stent holding portion that is fixed to the core shaft at a position between the coil body and the pusher portion. In the pusher guide wire, frictional resistance between the stent and the stent holding portion is larger at a proximal end portion of the stent holding portion than at a distal end portion of the stent holding portion.

### CROSS REFERENCE TO RELATED APPLICATION

This application claims priority to Japanese Application No. 2014-207392 filed on Oct. 8, 2014, the content of which is incorporated by reference herein in its entirety.

### BACKGROUND OF THE INVENTION

The disclosed embodiments relate to a medical device. Specifically, the disclosed embodiments relate to a pusher guide wire for delivering a stent housed in a catheter to a target site.

A stent is a piece of medical equipment used to treat a stricture or a disorder that has developed in a blood vessel or a digestive organ. For example, a stent can be used to support a lumen of a blood vessel or a digestive organ that has been expanded from a constricted condition in order to prevent the blood vessel or the digestive organ from again becoming constricted, or can be used to confine an embolus coil within an aneurysm formed in an arterial blood vessel of the abdomen or the brain so that the aneurysm does not rupture.

In general, a stent may be categorized as either a balloon-expandable stent that is expanded by a balloon catheter, or a self-expandable stent that expands spontaneously when no longer constricted. In recent years, self-expandable stents that do not easily become deformed under external force are greatly used.

A known method of delivering a self-expandable stent to a target site involves releasing the stent to the target site from a distal end of the catheter by pushing a pusher guide wire in the distal direction while the stent is held by a stent holding portion of the pusher guide wire. See, for example, Japanese Patent No. 4498709 and Japanese Unexamined Patent Application Publication No. 2013-521022 (Translation of PCT Application).

However, in conventional pusher guide wires, the frictional resistance between the stent and the stent holding portion is the same at the distal end of the stent holding portion as at the proximal end of the stent holding portion. Accordingly, even when a technician that has started to release the stent to the target site from the distal end of the catheter notices that the delivering position of the stent is displaced from the target site and pulls the pusher guide wire in the proximal direction, because the frictional resistance between the stent and the stent holding portion is small, the stent disadvantageously cannot be retrieved into the catheter (in other words, the procedure cannot be redone). Accordingly, when delivering the stent to the target site from the distal end of the catheter, the technician needs to manipulate the stent in a cautious manner.

### SUMMARY

The disclosed embodiments have been devised in view of the above circumstances and aim to provide a pusher guide wire that is capable of retrieving a stent, which is in the course of being released from a distal end of a catheter, into the catheter once more by setting the frictional resistance between the stent and a stent holding portion to be larger at a proximal end of the stent holding portion than at a distal end of the stent holding portion.

The above problem is addressed by the pusher guide wires described below.

A pusher guide wire for delivering a stent to a target site includes a core shaft, a coil body that covers a distal end portion of the core shaft, a pusher portion that is fixed to the core shaft at a position proximal to the coil body, and a stent holding portion that is fixed to the core shaft at a position between the coil body and the pusher portion.

A frictional resistance between the stent and the stent holding portion of the pusher guide wire is larger at a proximal end portion of the stent holding portion than at a distal end portion of the stent holding portion. Since the frictional resistance between the stent and the stent holding portion is smaller at the distal end portion of the stent holding portion, a risk of the stent being caught by the stent holding portion is reduced when the stent is released to the target site from the distal end of the catheter. Additionally, since the frictional resistance between the stent and the stent holding portion is larger at the proximal end portion of the stent holding portion, when the technician notices that the delivering position of the stent is displaced from the target site and pulls the pusher guide wire in the proximal direction, the stent in the course of being released can be retrieved into the catheter (in other words, the procedure can be redone). Furthermore, since the frictional resistance between the stent and the stent holding portion is smaller at the distal end portion of the stent holding portion, when the stent is retrieved into the catheter, a risk of the stent coming into contact with the stent holding portion and becoming deformed is reduced; accordingly, the technician can repeatedly retrieve the stent into the catheter until the delivery position of the stent coincides with the target site.

### BRIEF DESCRIPTION OF THE DRAWINGS

FIGS. 1A to 1C are diagrams each illustrating an entirety of a pusher guide wire of the disclosed embodiments. FIG. 1A is a diagram illustrating a state in which a stent is housed in a catheter. FIG. 1B is a diagram illustrating a state in which the pusher guide wire is pushed in a distal direction such that the stent is in the course of being released from a distal end of the catheter. FIG. 1C is a diagram illustrating a state in which the pusher guide wire is pulled in a proximal direction such that the stent, which is in the course of being released, is retrieved into the catheter.

FIGS. 2A to 2C are diagrams each illustrating an entirety of a pusher guide wire of the disclosed embodiments. FIGS. 2A to 2C correspond to FIGS. 1A to 1C, respectively.

FIGS. 3A to 3C are diagrams each illustrating an entirety of a pusher guide wire of the disclosed embodiments. FIGS. 3A to 3C correspond to FIGS. 1A to 1C, respectively.

FIGS. 4A to 4C are diagrams each illustrating an entirety of a pusher guide wire of the disclosed embodiments. FIGS. 4A to 4C correspond to FIGS. 1A to 1C, respectively.

FIGS. 5A to 5C are each a first modification of FIGS. 4A to 4C, respectively.

FIGS. 6A to 6C are each a second modification of FIGS. 4A to 4C, respectively.

FIGS. 7A to 7C are diagrams each illustrating an entirety of a pusher guide wire of the disclosed embodiments. FIGS. 7A to 7C correspond to FIGS. 1A to 1C, respectively.

### DETAILED DESCRIPTION OF EMBODIMENTS

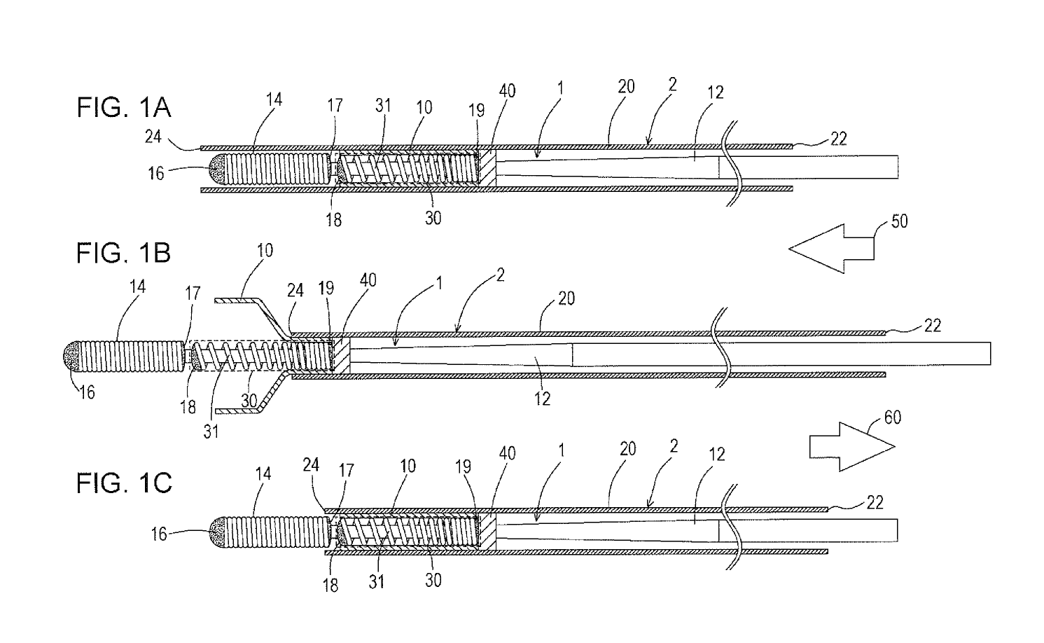

Referring first to FIGS. 1A to 1C, a pusher guide wire 1 of the disclosed embodiments will be described. In each of FIGS. 1A to 7C, the left side of each drawing corresponds to a distal end (a front end) of a catheter to be inserted into the body, and the right side of each drawing corresponds to a proximal end (a base end) of the catheter to be manipulated by a technician, such as a doctor.

As illustrated in FIGS. 1A to 1C, the pusher guide wire 1 for delivering a stent 10 to a target site includes a core shaft 12, a first coil body 14 (a distal end coil body) that covers a distal end portion of the core shaft 12, a first securing portion 16 that joins a distal end of the core shaft 12 and a distal end of the first coil body 14 to each other, a pusher portion 40 that is disposed on the proximal side (the right side in the drawing) with respect to the first coil body 14 and that is fixed to the core shaft 12, and a stent holding portion 30 that is disposed between the first coil body 14 and the pusher portion 40 and that is formed of a second coil body 31 that is fixed to the core shaft 12.

A proximal end of the first coil body 14 is joined to the core shaft 12 with a second securing portion 17. Furthermore, a distal end of the second coil body 31 is joined to the core shaft 12 with a third securing portion 18, and a proximal end of the second coil body 31 is joined to the core shaft 12 with a fourth securing portion 19.

A catheter 2 includes a cylindrical body 20 into which the pusher guide wire 1 can be inserted. The pusher guide wire 1 that holds the stent 10 on the outer periphery of the stent holding portion 30 is inserted through a proximal end opening 22 of the catheter 2, and by pushing the core shaft 12 in a distal direction (the direction of arrow 50), the stent 10 can be released to the target site through the distal end opening 24 of the catheter 2. Note that since a known catheter can be used, a detailed description of the catheter 2 is omitted.

Referring to FIGS. 1A to 1C, a method of delivering the stent 10 that is housed in the catheter 2 to the target site with the pusher guide wire 1 will be described next.

The technician inserts the catheter 2 so that the distal end opening 24 of the catheter 2 coincides with the target site. While the catheter 2 is fixed at the target site so as not to move, the pusher guide wire 1 is inserted through the proximal end opening 22 of the catheter 2, and the core shaft 12 is pushed in the distal direction (the direction of arrow 50) so that the stent 10 is housed in the vicinity of the distal end opening 24 of the catheter 2 (see FIG. 1A). After checking again whether the distal end opening 24 of the catheter 2 coincides with the target site, the core shaft 12 is pushed further in the distal direction such that the stent 10 is released to the target site through the distal end opening 24 of the catheter 2 (see FIG. 1B).

However, in a case in which a distal end portion of the catheter 2 is bent because the target site is located in a bent peripheral blood vessel, when the core shaft 12 is pushed hard in the distal direction, the distal end opening 24 of the catheter 2 may disadvantageously move in the distal direction such that the release position of the stent 10 is disadvantageously displaced from the target site. Furthermore, in the course of releasing the stent 10 through the distal end opening 24 of the catheter 2, there may be a case in which one notices that the release position of the stent 10 is displaced from the target site. Furthermore, when the stent 10 is released through the distal end opening 24 of the catheter 2, the stent 10 expands in the radial direction and, at the same time, becomes shorter in the axial direction. Accordingly, when the lumen of the blood vessel or the digestive organ is larger than expected, the release position of the stent 10 may be displaced from the target site. In each of the above cases, the stent 10 that is in the course of being released needs to be retrieved into the catheter 2, and the procedure needs to be redone.

As illustrated in FIGS. 1A to 1C, in the pusher guide wire 1, the stent holding portion 30 is formed of the second coil body 31, and pitch intervals of the second coil body 31 are smaller towards the proximal end of the second coil body 31 than towards the distal end of the second coil body 31. With this configuration, the frictional resistance between the stent 10 and the stent holding portion 30 is larger towards the proximal end of the stent holding portion 30 than towards the distal end of the stent holding portion 30. Since the frictional resistance between the stent 10 and the stent holding portion 30 is larger towards the proximal end of the stent holding portion 30, when noticing that the delivery position of the stent 10 is displaced from the target site, the technician can retrieve the stent 10, which is in the course of being released, into the catheter 2 by pulling the core shaft 12 in a proximal direction (a direction of arrow 60), and can redo the procedure (see FIG. 1C).

Furthermore, in the pusher guide wire 1, since the frictional resistance between the stent 10 and the stent holding portion 30 is smaller towards the distal end of the stent holding portion 30, when releasing the stent 10 to the target site through the distal end opening 24 of the catheter 2, the risk of the stent 10 being caught by the stent holding portion 30 is reduced. Furthermore, since the frictional resistance between the stent 10 and the stent holding portion 30 is smaller towards the distal end of the stent holding portion 30, when the stent 10 is retrieved into the catheter 2 as illustrated in FIG. 1C, the risk of the stent 10 coming into contact with the stent holding portion 30 and becoming deformed is reduced; accordingly, the technician can repeatedly retrieve the stent 10 into the catheter 2 until the delivery position of the stent 10 coincides with the target site.

As described above, the pusher guide wire I has a simple configuration in which the pitch intervals of the second coil body 31 are changed, enabling the stent 10 that is in the course of being released to be retrieved into the catheter 2 so that the procedure can be redone (see FIG. 1C).

The materials of the elements of the pusher guide wire 1 will be described; however, the materials are not particularly limited to the materials described.

The core shaft 12 may be formed of stainless steel (SUS 304, SUS316, or the like) or a super-elastic alloy, such as a Ni—Ti alloy.

The first coil body 14 and the second coil body 31 may each be formed of a radiopaque wire. The above may include, for example, gold, platinum, tungsten, or an alloy formed of these elements. By forming the first coil body 14 and the second coil body 31 with radiopaque wires, the technician can perceive the positions of the first coil body 14 and the second coil body 31 in a fluoroscopic image and, as a result, can perceive the position of the stent 10 housed in the cylindrical body 20 of the catheter 2.

Note that the first coil body 14 and the second coil body 31 may each be formed of single wire or may be formed of a twisted wire formed from a plurality of wires twisted together. Compared with a single wire, the twisted wire is superior in properties such as flexibility and restorability; accordingly, the first coil body 14 and the second coil body 31 are desirably formed of a twisted wire.

The securing portions 16, 17, 18, and 19 may be formed of a brazing material (aluminum alloy solder, silver solder, gold solder, or the like) or a metal solder (Au—Sn alloy or the like).

Similar to the core shaft 12, the pusher portion 40 may be formed of stainless steel (SUS 304, SUS316, or the like) or a super-elastic alloy, such as a Ni—Ti alloy.

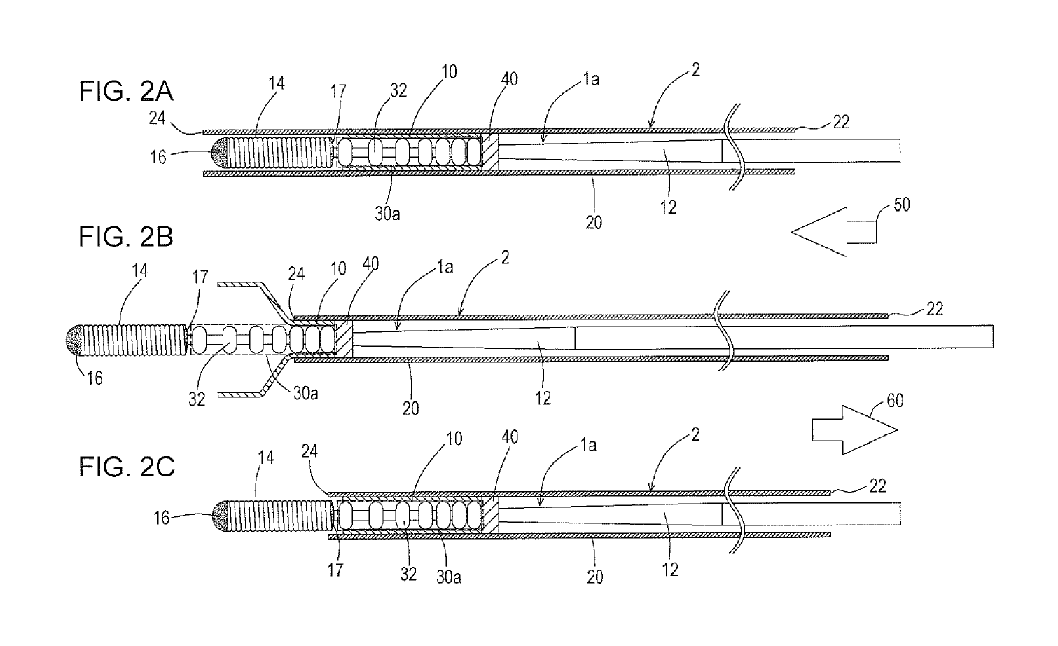

Referring to FIGS. 2A to 2C, a pusher guide wire 1a of the disclosed embodiments will be described next. Points that are different with respect to the pusher guide wire 1 illustrated in FIGS. 1A to 1C will be described.

In the pusher guide wire 1a, a stent holding portion 30a is formed of a plurality of bulging portions 32, and intervals between the bulging portions 32 are smaller towards a proximal end of the stent holding portion 30a than towards a distal end of the stent holding portion 30a. With this configuration, the frictional resistance between the stent 10 and the stent holding portion 30a is larger towards the proximal end of the stent holding portion 30a than towards the distal end of the stent holding portion 30a. Because the intervals between the bulging portions 32 are larger towards the distal end of the stent holding portion 30a, the frictional resistance between the stent 10 and the plurality of bulging portions 32 is smaller towards the distal end of the stent holding portion 30a. Likewise, because the intervals between the bulging portions 32 are smaller towards the proximal end of the stent holding portion 30a, the frictional resistance between the stent 10 and the plurality of bulging portions 32 is larger towards the proximal end of the stent holding portion 30a. As a result, when noticing that the delivery position of the stent 10 is displaced from the target site, the technician can retrieve the stent 10, which is in the course of being released, into the catheter 2 by pulling the core shaft 12 in the proximal direction (the direction of arrow 60), and can redo the procedure once again (see FIG. 2C).

As described above, the pusher guide wire 1a has a simple configuration in which the intervals of the plurality of bulging portions 32 are changed, enabling the stent 10 that is in the course of being released to be retrieved into the catheter 2 so that the procedure can be redone (see FIG, 2C).

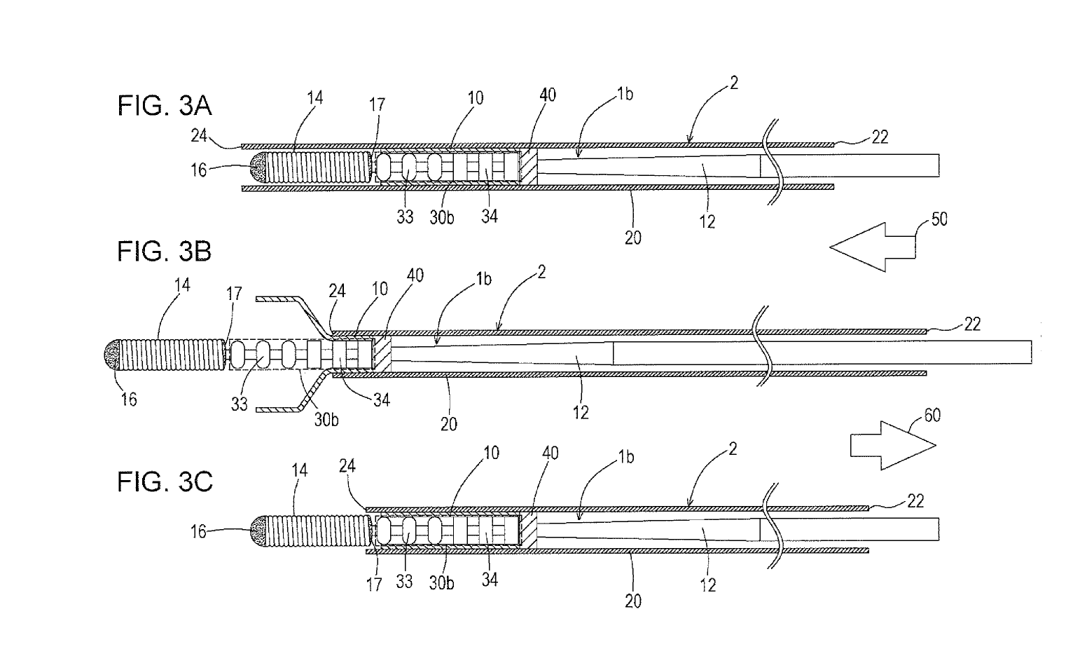

Referring to FIGS. 3A to 3C, a pusher guide wire 1b of the disclosed embodiments will be described next. Points that are different with respect to the pusher guide wire 1a illustrated in FIGS. 2A to 2C will be described.

In the pusher guide wire 1b, a first plurality of bulging portions 33 are formed towards a distal end of a stent holding portion 30b (at a distal end portion), while a second plurality of bulging portions 34 are formed towards a proximal end of the stent holding portion 30b (at a proximal end portion). Each of the first plurality of bulging portions 33 and each of the second plurality of bulging portions 34 are fixed to the core shaft 12 at a regular interval. The frictional resistance between the first plurality of bulging portions 33 and the stent 10 is small, while the frictional resistance between the second plurality of bulging portions 34 and the stent 10 is large. This can be accomplished, for example, if a shape of the first plurality of bulging portions 33 is different than a shape of the second plurality of bulging portions 34. With this configuration, the frictional resistance between the stent 10 and the proximal end portion of the stent holding portion 30b is larger than the frictional resistance between the stent 10 and the distal end portion of the stent holding portion 30b. As a result, when noticing that the delivery position of the stent 10 is displaced from the target site, the technician can retrieve the stent 10, which is in the course of being released, into the catheter 2 by pulling the core shaft 12 in the proximal direction (the direction of arrow 60), and can redo the procedure (see FIG. 3C).

As shown in FIGS. 3A-3C, the pusher guide wire lb has a configuration that uses the first plurality of bulging portions 33 and the second plurality of bulging portions 34 that have different shapes, thereby enabling the stent 10 that is in the course of being released to be retrieved into the catheter 2 and the procedure to be redone (see FIG. 3C).

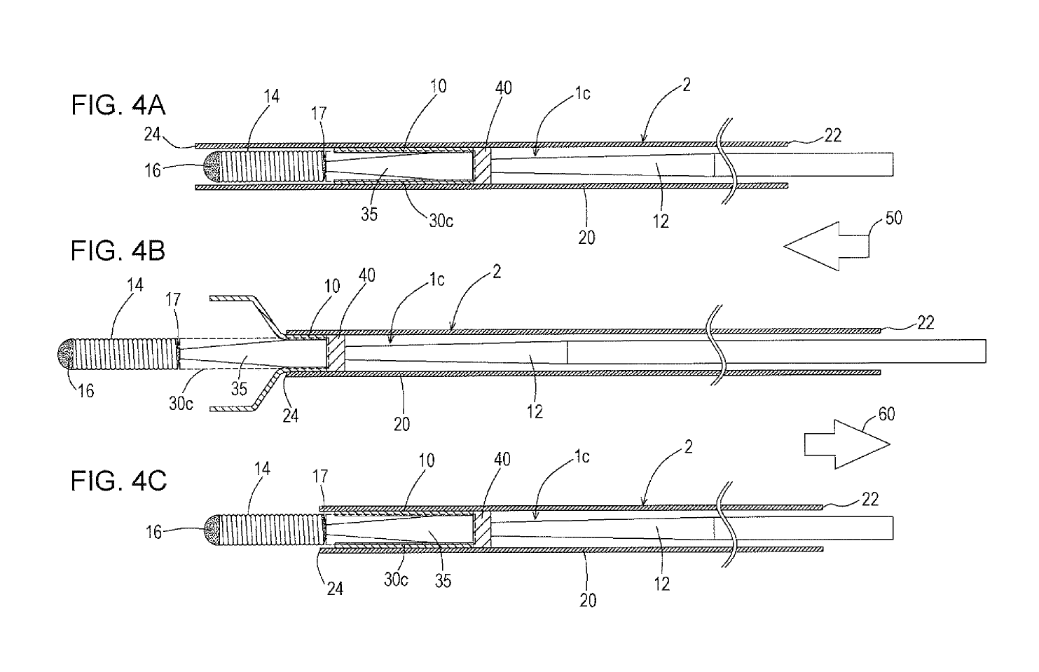

Referring to FIGS. 4A to 6C, a pusher guide wire 1c of the disclosed embodiments will be described next. Points that are different to the pusher guide wire 1 illustrated in FIGS. 1A to 1C will be described.

In the pusher guide wire 1c illustrated in FIGS. 4A to 4C, a stent holding portion 30c is formed of a tube body 35 of which an outer diameter is reduced from a proximal end of the stent holding portion 30e towards a distal end of the stent holding portion 30c. Accordingly, a gap between the stent 10 and the tube body 35 is smaller towards the proximal end of the stent holding portion 30c than towards the distal end of the stent holding portion 30c. With this configuration, the frictional resistance between the stent 10 and the stent holding portion 30c is larger towards the proximal end of the stent holding portion 30c than towards the distal end of the stent holding portion 30c. As a result, when noticing that the delivery position of the stent 10 is displaced from the target site, the technician can retrieve the stent 10, which is in the course of being released, into the catheter 2 by pulling the core shaft 12 in the proximal direction (the direction of arrow 60), and can redo the procedure. Furthermore, when the stent 10 is retrieved into the catheter 2, the risk of the stent 10 coming into contact with the stent holding portion 30c and becoming deformed can be further reduced; accordingly, the technician can repeatedly retrieve the stent 10 into the catheter 2 until the delivery position of the stent 10 coincides with the target site.

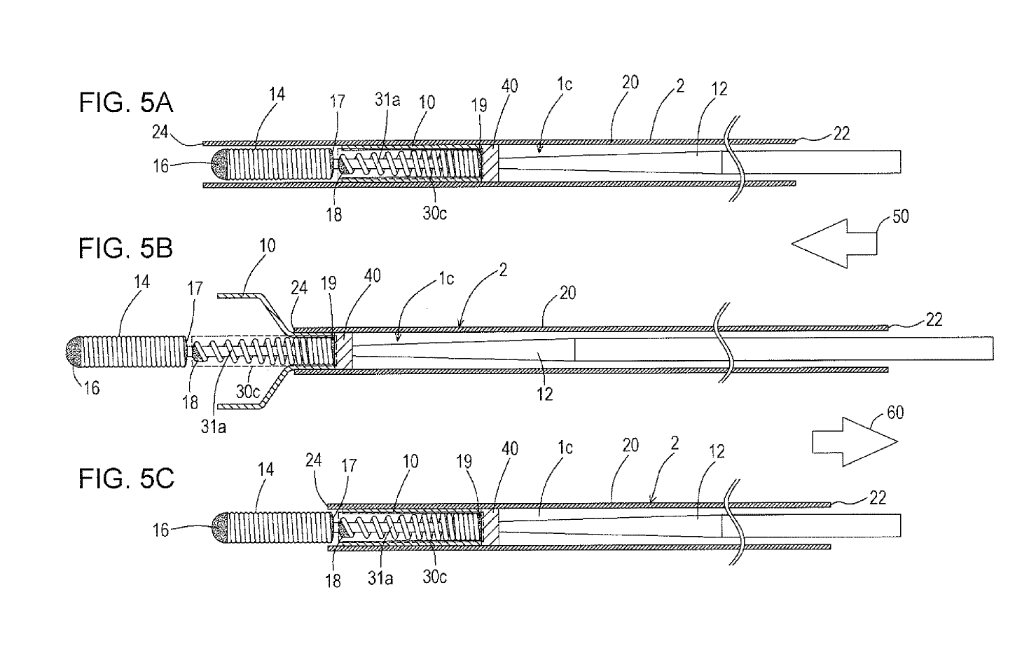

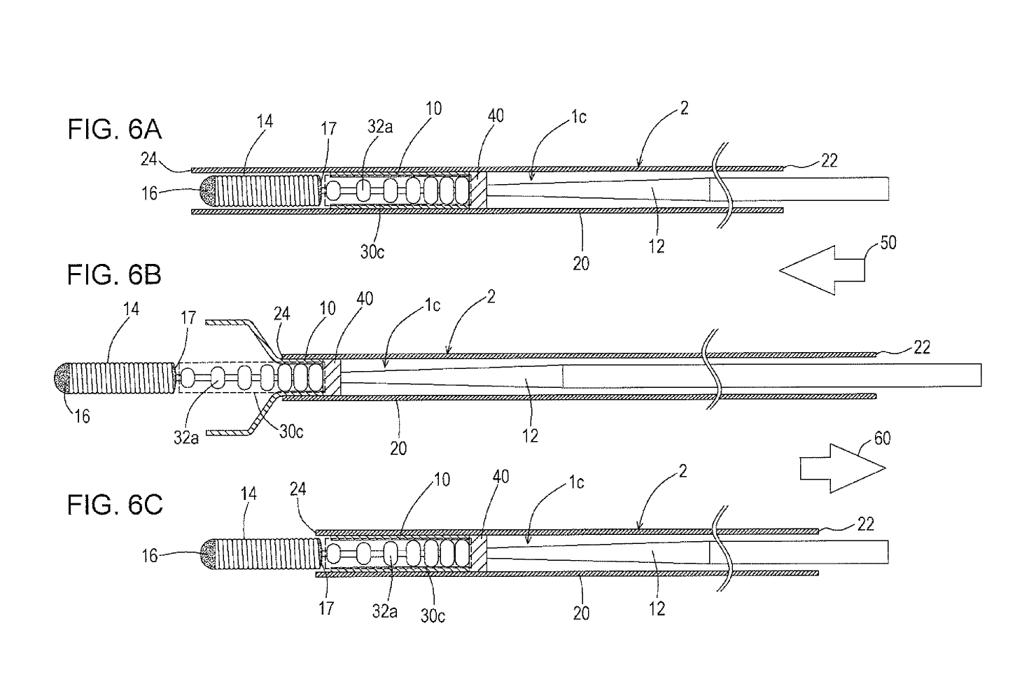

As a modification of the pusher guide wire 1c shown in FIGS. 4A to 4C, and as illustrated in FIGS. 5A to 5C, the stent holding portion 30c may be formed of a second coil body 31a of which an outer diameter is reduced from a proximal end of the stent holding portion 30c towards a distal end of the stent holding portion 30c. Furthermore, as illustrated in FIGS. 6A to 6C, the stent holding portion 30c may be formed of a plurality of bulging portions 32a having a circular cross section and whose diameters are reduced from the proximal end of the stent holding portion 30c towards the distal end of the stent holding portion 30c.

As described above, the pusher guide wire 1c has a configuration using either the tube body 35 whose outer diameter is reduced from the proximal end of the stent holding portion 30c towards the distal end of the stent holding portion 30c, the second coil body 31a whose outer diameter is reduced from the proximal end of the stent holding portion 30c towards the distal end of the stent holding portion 30c, or the plurality of bulging portions 32a whose diameters are reduced from the proximal end of the stent holding portion 30c towards the distal end of the stent holding portion 30c. Thus, when the stent 10 is retrieved into the catheter 2, the pusher guide wire 1c can reduce the risk of the stent 10 coming into contact with the stent holding portion 30c and becoming deformed (see FIGS. 4C, 5C, and 6C).

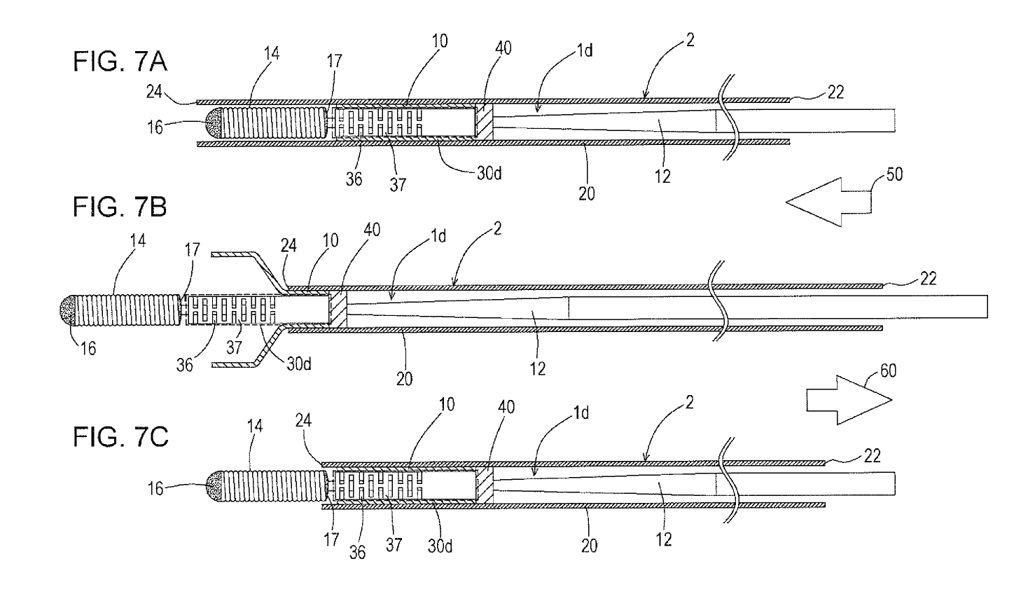

Referring to FIGS. 7A to 7C, a pusher guide wire 1d of the disclosed embodiments will be described. Points that are different with respect to the pusher guide wire 1 illustrated in FIGS. 1A to 1C will be described.

In the pusher guide wire 1d, a stent holding portion 30d is formed of a columnar tube body 37. A distal end portion of the columnar tube body 37 is provided with grooves 36, while a proximal end portion of the columnar tube body 37 contains no grooves 36. With this configuration, the frictional resistance between the stent 10 and the stent holding portion 30d is larger at the proximal end portion of the stent holding portion 30d than at the distal end portion of the stent holding portion 30d. As a result, when noticing that the delivery position of the stent 10 is displaced from the target site, the technician can retrieve the stent 10, which is in the course of being released, into the catheter 2 by pulling the core shaft 12 in the proximal direction (the direction of arrow 60), and can redo the procedure (see FIG. 7C).

As described above, in the pusher guide wires 1, 1a, 1b, 1c, and 1d, the frictional resistance between the stent 10 and the stent holding portions 30, 30a, 30b, 30c, and 30d is larger at the proximal end portion than at the distal end portion of the stent holding portions 30, 30a, 30b, 30c, and 30d. Thus, when noticing that the delivery position of the stent 10 is displaced from the target site, the technician can retrieve the stent 10, which is in the course of being released, into the catheter 2 by pulling the core shaft 12 in the proximal direction (the direction of arrow 60), and can redo the procedure. Furthermore, since the frictional resistance between the stent 10 and the stent holding portions 30, 30a, 30b, 30c, and 30d is smaller at the distal end portion of the stent holding portions 30, 30a, 30b, 30c, and 30d, there is a reduced risk of the stent 10 being caught by the stent holding portions 30, 30a, 30b, 30c, and 30d when releasing the stent 10 to the target site through the distal end opening 24 of the catheter 2. Furthermore, since the frictional resistance between the stent 10 and the stent holding portions 30, 30a, 30b, 30c, and 30d is smaller at the distal end portion of the stent holding portions 30, 30a, 30b, 30c, and 30d, there is a reduced risk of the stent 10 coming into contact with the stent holding portions 30, 30a, 30b, 30c, and 30d and becoming deformed when the stent 10 is retrieved into the catheter 2; accordingly, the technician can repeatedly retrieve the stent 10 into the catheter 2 until the delivery position of the stent 10 coincides with the target site.

What is claimed is:

1. A pusher guide wire for delivering a stent to a target site, the pusher guide wire comprising:
a core shaft;
a first coil body that covers a distal end portion of the core shaft;
a pusher portion that is fixed to the core shaft at a position proximal to the first coil body; and
a stent holding portion that is fixed to the core shaft at a position between the first coil body and the pusher portion,
wherein frictional resistance between the stent and the stent holding portion is larger at a proximal end portion of the stent holding portion than at a distal end portion of the stent holding portion.

2. The pusher guide wire according to claim 1, wherein:
the stent holding portion is formed of a second coil body, and
pitch intervals of the second coil body are smaller towards a proximal end of the second coil body than towards a distal end of the second coil body.

3. The pusher guide wire according to claim 1, wherein:
the stent holding portion is formed of a plurality of bulging portions, and
intervals between the plurality of bulging portions are smaller towards a proximal end of the stent holding portion than towards a distal end of the stent holding portion.

4. The pusher guide wire according to claim 1, wherein:
a diameter of the stent holding portion is reduced from a proximal end of the stent holding portion towards a distal end of the stent holding portion, and
a gap between the stent and the stent holding portion is smaller towards the proximal end of the stent holding portion than towards the distal end of the stent holding portion.

5. The pusher guide wire according to claim 2, wherein:
an outer diameter of the second coil body is reduced from the proximal end of the second coil body towards the distal end of the second coil body, and
a gap between the stent and the second coil body is smaller towards the proximal end of the second coil body than towards the distal end of the second coil body.

6. The pusher guide wire according to claim 3, wherein:
a diameter of the stent holding portion is reduced from the proximal end of the stent holding portion towards the distal end of the stent holding portion, and
a gap between the stent and the stent holding portion is smaller towards the proximal end of the stent holding portion than towards the distal end of the stent holding portion.

7. The pusher guide wire according to claim 1, wherein:
the stent holding portion is formed of a first plurality of bulging portions located at a distal end portion of the stent holding portion and a second plurality of bulging portions located at a proximal end portion of the stent holding portion, and
frictional resistance between the stent and the first plurality of bulging portions is smaller than frictional resistance between the stent and the second plurality of bulging portions.

8. The pusher guide wire according to claim 7, wherein a shape of the first plurality of bulging portions is different than a shape of the second plurality of bulging portions.

9. The pusher guide wire according to claim 1, wherein:
the stent holding portion is formed of a tube body,
an outer diameter of the tube body is reduced from a proximal end of the tube body towards a distal end of the tube body, and
a gap between the stent and the tube body is smaller towards the proximal end of the tube body than towards the distal end of the tube body.

10. The pusher guide wire according to claim 1, wherein:
the stent holding portion is formed of a columnar tube body,
a distal end portion of the columnar tube body comprises grooves, and
a proximal end portion of the columnar tube body has no grooves.
